(19) United States
(12) Patent Application Publication
(10) Pub. No.: US 2022/0338465 A1
(43) Pub. Date: Oct. 27, 2022

(54) FLUIDIC PLATFORMS FOR PERFUSABLE VASCULARIZED TISSUES

(71) Applicant: Massachusetts Institute of Technology, Cambridge, MA (US)

(72) Inventors: Roger Kamm, Cambridge, MA (US); Zhengpeng Wan, Cambridge, MA (US); Shun Zhang, Cambridge, MA (US)

(21) Appl. No.: 17/728,642

(22) Filed: Apr. 25, 2022

Related U.S. Application Data
(60) Provisional application No. 63/179,041, filed on Apr. 23, 2021.

(51) Int. Cl. A01N 1/02

(52) U.S. Cl. CPC A01N 1/0247; A01N 1/021

(57) ABSTRACT

Microfluidic platforms for forming and culturing perfusable hydrogel vascularized tissues typically include one or more culture chambers. Each culture chamber includes at least two openings overlaid over a gel channel. The gel channel typically includes at least two tissue zones and a trapping or insertion portion positioned between the tissue zones. The trapping or insertion portion permits vascular networks to develop between the two tissue zones containing vascularized tissues and/or vascularized tissue masses. The vascularized tissue masses in the tissue zones of the gel channel are connected indirectly, via the vascular network of the trapping portion. Also described are methods of forming and culturing perfusable vascularized tissue masses directly or indirectly interconnected via vascularized networks.

### CROSS-REFERENCE TO RELATED APPLICATIONS

This application claims priority to and benefit of U.S. Provisional Application No. 63/179,041, filed Apr. 23, 2021, which is hereby incorporated herein by reference in its entirety.

### STATEMENT REGARDING FEDERALLY SPONSORED RESEARCH

This invention was made with Government support under Grant No. U01 CA214381 awarded by the National Institutes of Health (NIH). The Government has certain rights in the invention.

### FIELD OF THE INVENTION

This invention is generally directed to microfluidic devices supporting tissue perfusion and vascularization.

### BACKGROUND OF THE INVENTION

Besides participating in metabolic function, microvasculature has unique biological functions and physical properties, such as maintaining solute and water balance between the blood and tissue compartments, and responding to different deformations and stress fluctuations (Jain et al., Nat. Med., 9:685-693 (2003)). Recently, the concept of “organ-on-a-chip” has been proposed to establish in vitro models that can mimic the microphysiological function and three-dimensional (3D) microstructure of human organ more accurately and specifically compared to the traditional two-dimensional (2D) cultures and animal models (Bhatia et al., Nat. Biotechnol., 32:760-772 (2014)). In addition to supplying nutrients and oxygen to the cultured tissue by perfusing the culture medium, vascularization of an organ-on-a-chip can also contribute to the establishment of organ-specific microenvironments and microphysiological function by constructing the microvascular with selective barrier function similar to that in vivo. Accordingly, there is a need for a perfusable and functional 3D microvasculature applicable to different organ-on-a-chip systems to better mimic the characteristics and functions of specific human organs in vitro (Wang et al., Micromachines, 9, 493:1-26 (2018)).

Microfluidic technologies have emerged as useful tools for the development of organ-on-a-chip, which can help control various aspects of the cellular microenvironment such as a different profile of fluid flow, gradient of various growth factors, and mechanical properties of versatile biomaterials.

There is a great potential to model pathological conditions to study vascular-related diseases. Especially for cancer biology, the tumor vasculature plays a critical role in several key events in the metastatic cascade, such as intravasation and extravasation. Engineered microvessels can be well suited to the study of mechanisms of tumor growth and metastasis, drug screening, and cancer therapies by establishing the vascularized microtumor models in vitro (Wang et al., Micromachines, 9, 493:1-26 (2018)). However, platforms for forming such vascularized tissue models in vitro, and providing tissues interconnected by the vascular networks, are not well developed.

Recent decades have witnessed an explosive growth in the number and variety of engineered microvascularized networks (“MVNs”). Functional vasculature is critical for the development of many microphysiological models and for diverse applications in tissue engineering. Although vasculature plays many important roles beyond just serving as conduits, its major function is still to transport nutrients, oxygen, metabolic wastes and various cells. Lacking a perfusable vasculature hinders the advancement of numerous key techniques for bioengineering. One particular example is organoid technology. Without a perfusable vasculature, organoids in 3D culture rely solely on passive diffusion to exchange nutrients, oxygen and metabolic waste, and gradually leads to a necrotic core. Organoids, especially originated from ectoderm, are largely avascular. Recently, there are some achievements in organoid vascularization in vitro by exogenously expressing ETV2 or introducing flow, however, the vasculatures of these organoids are only observed within the organoids but not connected to a perfusable vascular network. Some studies form perfusable tumor spheroids by injecting them into animal models. However, the reliance on an animal host limits both the scalability and the translation of organoid-based approaches, particularly for in vitro applications. Besides, the established perfusable organ-on-chip models mainly use artificial channels, or biocompatible membrane coated with the single type of endothelial cell (“ECs”) monolayers to serve as circulatory vasculature.

Various source of ECs and stromal cells have been used to mimic specific in vivo vascular microenvironment. Different types of ECs, or same type of ECs from different donors, are associated with varied levels of vasculogenesis ability. As a result, it is often difficult to reproduce those MVNs to compare results between labs.

It is therefore an object of the present invention to provide a method and devices to standardize techniques to ensure robust formation of functional MVNs with myriad sources of ECs. This is especially important with certain engineered organotypic MVNs that are difficult to form, or not available at this moment, which would save tremendous energy and resources from purchasing and testing different batches of relevant cells. Moreover, MVNs are often engineered to study interactions with other physiological systems, like neural or muscular, or used as platform to host various developing organoids. In those multi-culture systems, some crucial factors of the microenvironment, such as the choice of hydrogel scaffold and composition of culture medium, must be carefully designed to support the development and growth of all the cellular components. This poses a strong challenge for ECs since they must be able to self-organized into functional MVNs under suboptimal conditions.

Tremendous efforts have been made to construct functional vasculature in vitro within various hydrogels with associated microfluidic chips. Many of those were designed following self-organizing approach, which draw upon natural self-organizing and self-assembly principles by promoting conditions in which vasculogenesis or angiogenesis, two major ways of forming and developing vascularization in vivo, can occur. The major advantage of self-organizing method is its similarity to the in vivo processes to grow and develop vasculature, thus leading to spontaneous formation of vessels mimicking in vivo counterparts in both function and morphology. However, the inconsistency from the self-organizing nature and the cells in their ability to form functional MVNs hinder its wider application and limit broader impact. The majority of the engineered MVNs with perfusability were constructed from different sources of primary endothelial and stromal cells. Although various methods have been proposed to reduce the variability, including standardizing cell sources with iPSC or immortalized cells, improved seeding strategies, or identifying key growth factors in neovessel formation, an easily applicable approach that can effectively boost the vasculogenic ability of ECs and aid the robust formation of functional MVN is still in urgent need for the community.

A developing vasculature in vivo is subjected to various mechanical cues that were not incorporated in most in vitro MVN models, especially the shear stresses induced by pulsatile or unidirectional blood flow, transmural flow and interstitial flow (“IF”), which plays a critical role during vasculogenesis to drive nutrients, remove metabolic wastes, and provide mechanical cues to cells, before perfusable vasculature are established. Interstitial flow plays an important role in tissue morphogenesis, function, and pathology. It is most prominently linked to the drainage of blood plasma leaking from capillaries toward lymphatic vessels. Interstitial flow occurs through the extracellular matrix. In tissues with little extracellular matrix or in model tissues lacking an extracellular matrix, interstitial flow is controlled by permeability at the intercellular junctions which depends on the strength of intercellular adhesions.

Cellular aggregate permeability can be characterized by microfluidics. By placing a cellular aggregate in a specially designed chamber in a microfluidic channel, an interstitial flow can be imposed through it. The aggregate permeability can be quantified by assuming the aggregate to behave as a porous medium following Darcy's law

Various microfluidic platforms have been engineered to investigate the effects of IF on ECs and vessel formation. Most of the work reported a beneficial role of IF on angiogenesis, vasculogenesis and 3D capillary morphogenesis in vitro. Those evidence suggest IF would benefit the formation of self-organized MVN, which is validated recently by studies using generic MVN with Human umbilical vein endothelial cells (“HUVECs”) and human lung fibroblasts (“HLFs”) coculture, and brain specific MVN with triculture of Brain ECs, pericytes (“PCs”) and astrocytes (“AC”). However, those studies failed to maintain a relatively stable IF during neovessel formation, and did not investigate the effect of varied IF speeds. Moreover, the underlying mechanism leading to the distinct behaviors was not examined.

There remains a need for platforms supporting formation and culture of perfusable vascularized tissues biomimicking in vitro vascularized microtissue models.

Therefore, it is an object of the present invention to provide microfluidic platforms for forming and culturing perfusable vascularized tissues.

It is a further object of the present invention to provide microfluidic platforms for forming and culturing interconnected perfusable vascularized tissues, interconnected by vascular networks.

It is another object of the present invention to provide methods of forming microfluidic platforms for perfusable vascularized tissues.

It is yet another object of the present invention to provide methods of using microfluidic platforms to form and study perfusable vascularized tissues.

### SUMMARY OF THE INVENTION

A microfluidic platform for perfusable tissue culture, the platform including a variety of geometrical shapes of a gel-filled channel(s), through which an interstitial fluid (“IF”) is driven to support the formation of perfusable microvascular network, has been developed. The gel channel opens only to inlet and outlet, and has various designs to accommodate the inclusion of organoids, tumors and spheroids. The microfluidic platforms include one or more chambers having one or more interconnected conduits used to form and culture perfusable vascularized tissues and tissue masses. The chambers typically include a single gel channel overlaid by chamber openings. The gel channel includes at least two tissue zones and one or more trapping or insertion portions positioned between the tissue zones. The chamber openings typically contact the tissue zones of the gel channel. A fluid-holding column may be connected to the chamber openings to create IF. Additionally or alternatively, tubing may be connected to the chamber openings. The tubing may also be connected to a pump controlling fluid flow rate, or gravity flow used, to push fluid (i.e., IF) through the tissue, to control morphology and porosity of the resulting tissue. Alternatively, the pump may be incorporated into the device to produce one integrated system

Methods of forming vascularized tissue and/or vascularized tissue masses in the microfluidic platforms include the steps of seeding a gel channel of the microfluidic platforms with endothelial cells or a mix of cells and one or more extracellular matrix (“ECM”) components, and flowing medium through the gel channel to imitate the effect of interstitial fluid flow (“IF”). Typically, the mix of cells and one or more ECM components is seeded in one or both of the at least two tissue zones of the gel channel. The gel channel may be seeded with the same mix of cells and ECM components in each of its tissue zones or with a different mix of cells and ECM components. The cells typically form vascular networks throughout the gel channel, including the tissue zones and the trapping or inserting portions separating these tissue zones.

The cells typically include one or more cell types for forming vasculature, such as endothelial cells, stromal cells, smooth muscle cells, pericytes, fibroblasts, progenitor cells, and combinations thereof. The ECM components typically include one or more of fibrous proteins, hyaluronic acid, synthetic hydrogels, and proteoglycans. Suitable fibrous proteins include collagen, fibrin, fibronectin, elastins, and laminin. The mix of cells and ECM components may also include cells forming tissue masses, such as cells forming tumors, organoids, spheroids, and combinations thereof. The seeded mix of cells and ECM components are typically cultured for a period between about 2 and 10 days with culture medium flowing from one media port through the gel channel to a second media port.

The flow rate and pressure of the IF is used to manipulate the morphology and other properties of the resulting tissue masses. The examples demonstrate how the applied pressure and duration of application of IF affects formation of self-organized microvascular networks (“MVN”) in vitro. PCR analysis and inhibitory tests indicate that upregulation of MMP-2 boosts vasculogenesis ability of ECs induced by IF. Balancing MMP-2 activity through the interplay between IF and MMP-2 specific inhibitor, one can regulate many key morphological parameters of self-organized MVN, while maintaining its high perfusability.

MVNs initially were grown under various physiologically relevant IF in the single channel microfluidic chip, where IF was fully characterized by computation and experiments. After systematically investigating the beneficial role of IF on neovessel formation, it was demonstrated that MVN morphologies are determined by global IF rather than local IF. With a series of inhibitory experiments, it was established that the boosted vasculogenesis capacity of ECs results from upregulated MMP-2 when cultured under IF. Based on this mechanism, a method and system were developed based on the interplay between IF and MMP-2 inhibitor, for regulating many important morphological parameters of self-organized MVN, while maintaining perfusability and permeability. These findings were further validated with brain specific MVN made of brain endothelial cells (“BEC”), pericytes (“PC”), and astrocyte (“AC”) triculture to form a blood brain barrier type structure, establishing the mechanism and methodology to promote the formation of various functional MVNs, indicating the mechanism and methodology are generic to ECs.

The trapping or insertion portions in the gel channel are designed to place organoids, tumors, and spheroids for vascularization. Various types of organoids, for example, brain, liver, kidney, and heart organoids could be integrated to the microfluidic devices presented here. Tumor spheroids, either formed from cancer cell lines or patient derived tumor tissues are also compatible with these devices. Enhanced vascularization of these models was achieved using the devices and methods described here. Vascularized tissues could be further used for drug screening, immune cell perfusion, antibody uptake studies, etc.

### BRIEF DESCRIPTION OF THE DRAWINGS

FIGS. 1A-1H are diagrams showing details of different embodiments of microfluidic platforms for perfusable vascularized tissue growth.

FIGS. 2A-2C are prospective view showing the assembly of microfluidic platforms for perfusable vascularized tissue growth with a trapping zone in each channel. FIG. 2D is a prospective view of microfluidic platform 90. FIG. 2E is a schematic of microfluidic platform 90 where the one or more chambers 46a-46c are interconnected with tubing 94, which is in turn connected to a pump via connection 96, to establish perfusion through multiple tissue systems.

FIGS. 3A-3D are prospective views showing the microfluidic platforms for perfusable vascularized tissue growth with the insertion zone without the top opening at the insertion region.

FIGS. 4A-4D are prospective views showing the microfluidic platforms for perfusable vascularized tissue growth with the insertion zone with the top opening at the insertion region.

FIGS. 5A-5G are schematics of formation of in vivo like MVN with IF setup. FIG. 5A is a prospective view of a 3-channel microfluidic device for culturing MVN statically. FIG. 5B is a schematic of experimental setup to maintain IF using a single channel device with affixed syringes. FIG. 5C is a graph of time (hours) evolution of IF speed (μm/sec) under 20 mm H₂O pressure difference, computed with analytical solution and measured matrix permeability of acellular fibrin gel. IF speed (μm/sec) decreased over time, but was restored by replenishing the culture medium at 24-h intervals. FIG. 5D is a graph showing matrix permeability (m²) of acellular fibrin gel over time (days). n=5. FIG. 5E is a graph of average IF speed (μm/sec) within acellular fibrin gel measured under 10, 20, 30 mm H₂O pressure differences over six days. Dashed lines correspond to calculated average IF speed with analytical solution. FIG. 5F is a graph of average IF speed of the first 24 hours after HUVEC and HLF mixture seeded in devices under various pressure differences. n=5 devices for each IF condition. Significance were calculated with two sample t tests. ***P<0.01. FIG. 5G is a graph of matrix permeability (m²) measured daily during the first 3 days after HUVEC and HLF mixture seeded in devices. n=15 devices.

FIGS. 6A-6F show that IF boosts the formation of functional MVN. Studies were done with control no flow (“static”), IF 10 mm, IF 20 mm, and IF 30 mm. FIG. 6A is a graph of vessel cover area (%); FIG. 6B is a graph of the number of branch points; FIG. 6C is a graph of vessel segment length (μm); FIG. 6D is a graph of vessel diameter (μm); FIG. 6E is a graph of perfusability (%). Perfusability of MVN formed under various IF speed, grey dashed line indicates 80% perfusability. n=26 devices for static condition, n=6 devices for each IF condition. FIG. 6F is a graph of vessel diameter (μm) Diameters of perfusable vessels in inlet, middle, outlet region of MVN formed in single channel devices under various IF conditions. n=4 devices in each IF condition. Statistical analysis of morphological parameters of perfusable vessels cultured under various IF speed. n=17 devices for static condition, n=6 devices for each IF condition. Significance were calculated with two sample t tests. ***P<0.01.

FIGS. 7A-7F show that IF affects global MVN structures. FIG. 7A is a schematic diagram of a modified single channel device used to generate inhomogeneous IF. FIG. 7B is a table of average IF speed in neck or middle ROIs of modified single channel device with corresponding pressure differences, calculated in COMSOL FIGS. 7D-7F show the statistical analysis of morphological parameters of MVN in middle or neck regions of modified single channel device under 10 mm or 20 mm H₂O pressure differences. n=2 devices for each IF condition, 4 ROIs in each device (2 in neck region and 2 in middle region, as shown in (d)). FIG. 7C is a profile of IF speed induced by 10 mm H₂O pressure difference, calculated in COMSOL with acellular fibrin gel permeability measured in the experiment, showing highest speed in connecting structures and lowed speeds in culture wells and center. FIG. 7D is a graph of vessel diameter (μm); FIG. 7E is a graph of vessel coverage (%); and FIG. 7F is a graph of the number of branch points. Significance was calculated with two sample t tests. ***P<0.01.

FIGS. 8A-8E are graphs showing that IF enhances the vasculogenesis ability of EC through upregulation of MMP-2. MVN was formed statically with HUVEC monoculture in 3-channel device MVN was formed with HUVEC monoculture in single channel device under IF induced by 10 mm H₂O pressure difference, or with various inhibitors added: 100 μM PAI-1, 200 U/ml aprotinin, 50 μM marimast, 50 μM NSC 405020, 100 μg/ml MMP-1 neutralizing antibody and 100 μg/ml control antibody. Samples were pooled at day 3. n=3 FIGS. 8A-8D are graphs showing the statistical analysis of morphological parameters and perfusability of perfusable vessels cultured with various concentration of ARP-100. IF induced by 10 mm H₂O pressure difference were applied to all cases. n=5 devices for control, n=3 devices for each inhibitor treated group. Significance was calculated with two sample t tests. ***P<0.01. FIG. 8E is a graph of vascular permeability measurements of 40 kDa dextran for perfusable MVNs formed under various conditions. n=3 devices for each group, 3 measurements were performed in each device at different ROIs.

FIGS. 9A-9C are graphs showing that IF enhances formation of fully perfusable brain microvascular network, comparing brain specific MVN formed statically or under IF induced by 10 mm H₂O pressure difference. FIG. 9A is a graph of vessel diameter (μm); FIG. 9B is a graph of perfusability (%), and FIG. 9C is a graph of vascular permeability (cm/sec). Statistical analysis of morphological parameters and perfusability of brain specific MVN formed statically or under IF. n=6 devices for static group, n=3 devices for IF group.

FIGS. 10A-10D are graphs showing that IF enhances formation of fully perfusable microvascular network made by HUVEC and HLF. FIGS. 10A-10D are Statistical analysis of morphological parameters and perfusability of perfusable vessels cultured with various concentration of ARP-100. n=6 devices for control, n=3 devices for each inhibitor treated group. Significance was calculated with two sample t tests. ***P<0.01.

### DETAILED DESCRIPTION OF THE INVENTION

### I. Definitions

As used herein, the term “microfluidic” refers to devices with dimensions of fluidic pathway elements for manipulating and controlling fluids, usually in the range of hundreds of microliters (10⁻⁶). The microfluidic devices typically include a channel or a portion of a channel with dimensions from 0.1 to 10's of millimeters.

As used herein, “microvascular” refers to the part of the circulatory system made up of minute vessels (such as venules or capillaries) that average less than 0.3 millimeters in diameter.

As used herein, a microvascular network (“MVN”) is defined as a perfusable microvasculature network developed in a self-organizing manner by embedded endothelial cells, or endothelial cells and corresponding supporting cells, within a hydrogel containing one or more extracellular matrix components such as fibrin, usually by co-culturing the endothelial cells with stromal cells such as fibroblasts and pericytes.

As used herein, interstitial flow (“IF”) is defined as the movement of fluid through extracellular matrix. The range of interstitial flow is usually between 0.1 and 10 μm/s, and is achieved through MVN using gravitational flow as a function of hydrostatic head pressure difference across the gel or using a pump to flow the media/liquid through the stromal region.

As used herein, the term “perfusable” refers to a hydrogel or tissue structure permitting flow of fluid through the tissue. The perfusable tissue is a tissue having vascular elements crossing through the tissue and passing through the tissue. Vascular elements include hollow structures, such as hollow lumens lined with endothelial cells, capillaries, blood vessels, etc. The vascular elements may include vascular networks.

As used herein, the term “vascular networks” refers to a network of vascular elements, such as a network of hollow structures, a network of hollow lumens lined with endothelial cells, network capillaries, a network of blood vessels, etc. Vascular networks may be within tissue masses, as well as outside of tissue masses. Tissue masses containing vascular networks are typically perfusable tissue masses.

As used herein, the term “tissue masses” refers to aggregates of cells self-assembling into three-dimensional masses. Tissue masses include tumors, spheroids, organoids, and other self-assembled masses. As used herein, “spheroids” typically refers to a cluster of cells from a cultured cell line. As used herein, “organoid” refers to cell clusters in extracellular matrix such as primary cells in an extracellular matrix (“ECM”). This can include tissues obtained by biopsy.

Recitation of ranges of values herein are merely intended to serve as a shorthand method of referring individually to each separate value falling within the range, unless otherwise indicated herein, and each separate value is incorporated into the specification as if it were individually recited herein.

Use of the term “about” is intended to describe values either above or below the stated value in a range of approx. +/−10%; in other embodiments the values may range in value either above or below the stated value in a range of approx. +/−5%.

### II. Microfluidic Platforms

Microfluidic platforms and methods of use thereof for forming and culturing perfusable vascularized tissue and/or perfusable vascularized tissue masses have been developed.

A. Microfluidic Platform Components

The platforms typically include one or more chambers. Each platform includes chamber openings overlaid over a gel-filled channel. The chamber openings are typically connected to one or more columns or to tubing providing culture medium to the chamber. Each gel channel contains fluid with one or more extracellular matrix factors therein. Fluid flow through the chamber is generally controlled by the hydraulic pressure of the culture medium, either using head pressure for gravity flow and/or a pump, as demonstrated by the examples. As shown in the examples, IF plays a major role in tissue morphology and perfusability.

The platforms may include a plurality of chambers, such as between 2 and 12 chambers per platform. The plurality of chambers may be used for culturing cells, tissues, and/or tissue masses. In some embodiments, the plurality of chambers may be used for culturing tissues and tissue masses of the same mix of cells and ECM components. The plurality of chambers may be used for culturing cells, tissues, tissue masses of different mixes of cells and ECM components.

1. Chamber Opening

The microfluidic platforms typically include at least one chamber. The microfluidic platforms may include a plurality of chambers, such as between about 2 and 12 chambers, between about 2 and 10 chambers, between about 2 and 6 chambers, 5 chambers, 4 chambers, 3 chambers, or 2 chambers. Two chambers connected via a gel channel are preferred.

The chamber(s) on the microfluidic platform typically have chamber openings connected to each other via a gel channel. The chamber openings may be of any shape, but typically are circular or rectangular. The chamber openings may have a diameter or width between about 1.5 mm and 25 mm, between about 1.5 mm and 15 mm, between about 1.5 mm and 10 mm, between about 1.5 mm and 7.5 mm, between about 1.5 mm and 5 mm, or about 4 mm.

2. Chamber Gel Channel

The gel channel typically includes at least two tissue zones and a trapping or insertion portion positioned between the at least two tissue zones.

Generally, each of the tissue zones contacts at least one of the chamber openings. In some embodiments, the chamber includes two chamber openings and one gel channel with two tissue zones, where a portion of each of the two tissue zones connects to one of the chamber openings.

Typically, the gel channel has a length between about 5 and 30 mm, between about 5 mm and 25 mm, between about 5 mm and 20 mm, or about 16 mm. Typically, the gel channel, at is widest, has a width between about 1 mm and 25 mm, between about 1 mm and 15 mm, or between about 1 mm and 10 mm. The height of the gel channel is between 0.15 mm to 2 mm.

The gel channel has a length that is typically between about 2 times and 50 times greater than the length of the trapping or insertion region. The gel channel width, at its widest, is typically at least two times greater than the width of the trapping portion, but can be smaller than the width of the insertion region.

i. Tissue Zone(s)

The entire region, including both of the tissue zones of the gel channel, are typically filled with a mix of the cells and ECM components. The vascular networks begin to develop here and pass through the trapping or insertion region. Once developed, the vascular networks interconnect all the regions in the gel channel, including the tissue zones, through the trapping or insertion region.

The tissue zones typically have a width and depth to accommodate cell, tissue, and tissue mass formation and culture. The tissue zones, at their widest, may have a width between about 1.5 mm and 25 mm, between about 1.5 mm and 15 mm, between about 1.5 mm and 10 mm, between about 1.5 mm and 7.5 mm, between about 1.5 mm and 5 mm, about 2.5 mm, or about 2 mm.

ii. Trapping Region

Typically, the at least two tissue zones of the gel channel are separated by a trapping portion. The trapping portion has dimensions that permits vascular network development and traps tissue masses when being injected into the gel channel. The width of the gel channel varies depending on use.

The trapping portion may have a width between about 50 μm and 900 μm, between about 50 μm and 800 μm, between about 50 μm and 700 μm, between about 50 μm and 600 μm, between about 50 μm and 500 μm, about 750 μm, or about 350 μm.

The trapping portion may have a length between about 50 μm and 900 μm, between about 50 μm and 800 μm, between about 50 μm and 700 μm, between about 50 μm and 600 μm, between about 50 μm and 500 μm, about 750 μm, or about 350 μm.

iii. Insertion Region

Alternatively, the at least two tissue zones might be separated by an insertion region. It can have an ‘open-top’ design that is accessible from the top via a top port over the gel region locally, through which a tissue mass (such as an organoid, spheroid or tumor biopsy) can be inserted. Alternatively, the region can have the ‘closed-top’ design with organoid, spheroid or tumor sample inserted from the gel loading port. The insertion can occur at the time of filling the tissue zones simply by injecting the tissue mass into the gel solution via the top port prior to gel polymerization or via the gel loading port and enables the co-development of vasculature and inserted tissue. Alternatively, tissue mass can be inserted into the insertion region via the top port after perfusable vasculature formed. In this case, the inserted tissue can be surrounded by perfusable vasculature from the side, or from both side and bottom. Multiple insertion regions can fit in one single gel channel, connected by tissue zones.

3. Fluidic Column or Pump

Generally, the microfluidic platform has a fluid column or pump connected to the chamber. Typically, the columns or pump contact the chamber openings of the chamber. The fluid flow through a chamber is typically controlled with hydraulic pressure of the culture medium. For example, interstitial flow may be applied by the hydraulic pressure of the culture medium at different heights (H) of the culture medium in the column, or by operating a pump that provides a pressure difference across the gel, as demonstrated in the examples.

The fluid flow through the independent chambers may be controlled such that each chamber in a plurality of chambers has a unique fluid flow rate. The fluid flow through the chambers may be controlled such that all the chambers in a plurality of chambers have the same or similar fluid flow rate. The plurality of chambers on the platform may be fluidically interconnected. This may be achieved with tubing connecting fluid flow of different chambers on the platform. This fluidically connects together the different cells, tissues, and/or tissue masses in different chambers of the platform.

B. Exemplary Microfluidic Platforms

Single Channel Microfluidic Devices Using Trapping

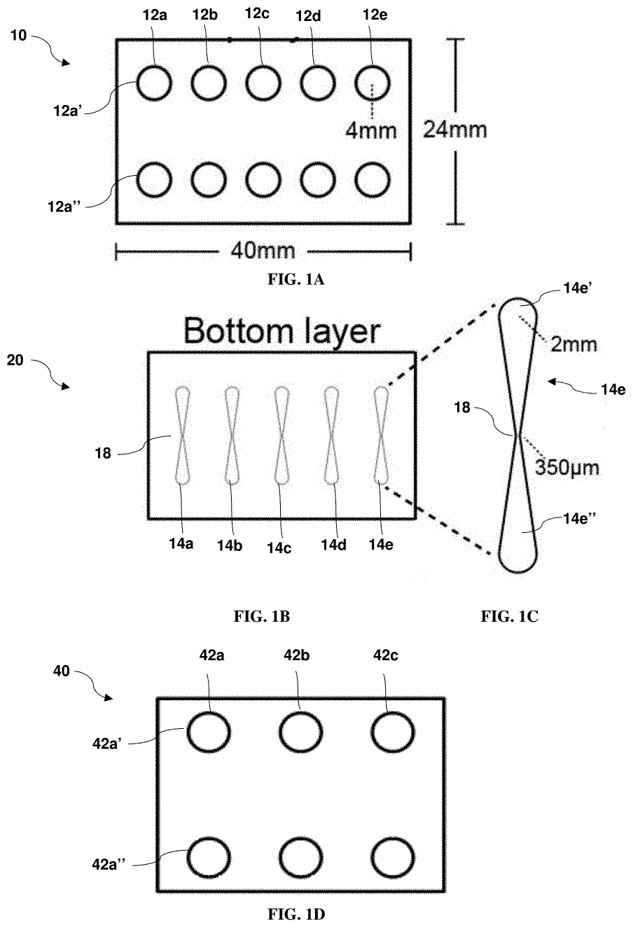

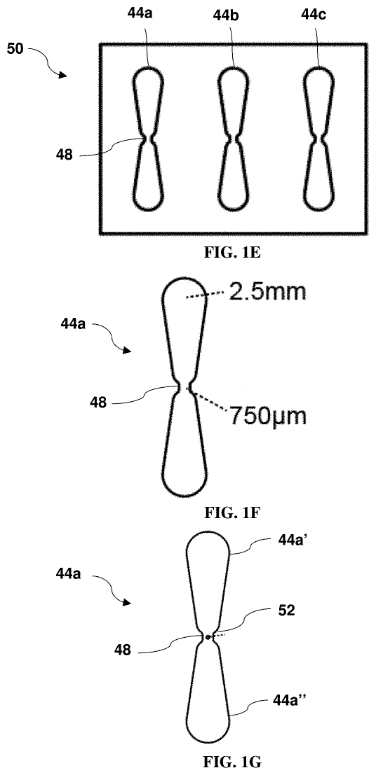

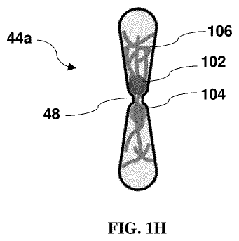

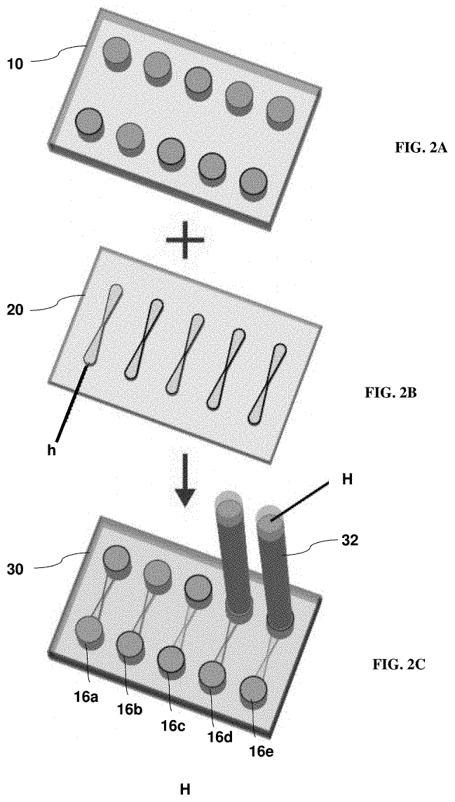

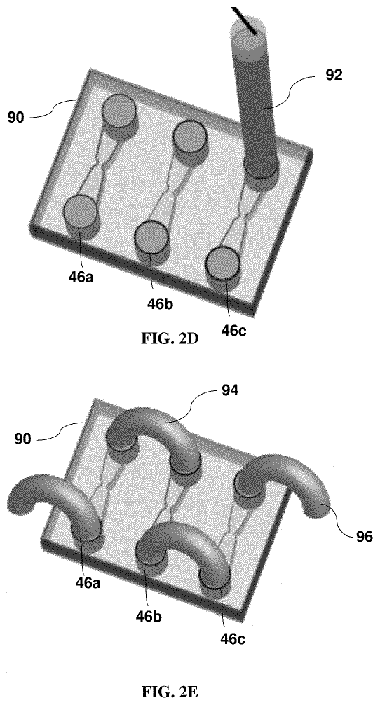

FIG. 1A shows a top layer 10 for the microfluidic platform 30 in FIG. 2C. The top layer 10 includes a plurality of opening pairs 12a-12e. Each opening pair includes two openings, such that the opening pair 12a include openings 12a′ and 12a″, and so on. FIG. 1B is a diagram of the bottom layer 20 for the microfluidic platform 30. The bottom layer 20 includes a plurality of gel channels 14a-14e.

The gel channels typically include at least two tissue zones separated by a narrow tissue trapping portion. The tissue trapping portion has dimensions that trap tumor, spheroid, or organoid when being seeded in the gel channel. The tissue trapping portion may be located at any point between the two tissue zones of the gel channel and may be between about 100 μm and about 1500 μm in width. Typically, the gel channel has a length at least two times greater than the length of the trapping portion. Generally, the gel channel has a length between about two times and 50 times greater than the length of the trapping portion. Generally, the tissue zones have a width between about two times and 30 times greater than the width of the trapping portion. FIG. 1C shows the tissue trapping portion 18 positioned between the two tissue zones 14e′ and 14e″ of the gel channel 14e. The gel channels are typically narrowest at the tissue trapping portion. The tissue trapping portion 18 is typically free of tumors, organoids or spheroids. The two tissue zones 14e′ and 14e″ of the gel channel 14e have a diameter of about 2 mm at their largest portions. In this embodiment, the tissue trapping portion 18 is located about the middle of the gel channel 14e and has a width of about 350 μm.

FIGS. 1D-1G are diagrams for another embodiment of a microfluidic platform. FIG. 1D shows the top layer 40 for the microfluidic platform 90 shown in FIG. 2D. The top layer 40 includes a plurality of opening pairs 42a-42c. Each opening pair includes two openings, such that the opening pair 42a include openings 42a′ and 42a″, and so on. FIG. 1E is a diagram of the bottom layer 50 for the microfluidic platform 90 of FIG. 2E. The bottom layer 20 includes a plurality of gel channels 44a-44c.

FIGS. 1F and 1G show the tissue trapping portion 48 positioned between the two tissue zones 44a′ and 44a″ of the gel channel 44a. The gel channels are typically narrowest at the tissue trapping portion, the vessel zone.

The tissue trapping portion 48 is typically free of organoids or spheroids. The two tissue zones 44a′ and 44a″ of the gel channel 44a have a diameter of about 2.5 mm at their largest portions. In this embodiment, the tissue trapping portion 48 is located about the middle of the gel channel 44a and has a width of about 750 μm. An opening 52 is positioned in the center of the tissue trapping portion 48 allows for air release from the tissues.

FIG. 1H is a diagram of the gel channel 44a with a tumor spheroid 102 in one tissue zone, with an organoid 104 in another tissue zone, the tumor spheroid 102 and the organoid 104 connected via the vascular networks 106 passing through the tissue trapping portion 48.

Each of the gel channels 14a-14e typically lines up with each one of the opening pairs 12a-12e to form the microfluidic platform 30, as shown in FIG. 2C.

FIGS. 2A-2E are diagrams showing the assembly of microfluidic platforms for perfusable vascularized tissue growth. FIGS. 2A-2C show the top layer 10 and the bottom layer 20 assemble together to form the microfluidic platform 30 with chambers 16a-16e. Each of the chambers 16a-16e includes opening pairs 12a-12e positioned over the gel channels chambers 14a-14e. The gel channels 14a-14e typically have a height (h) between about 10 μm and about 2000 μm. Hydraulic columns 32 may be connected to the one or more chambers 16a-16e to provide flow through the chamber(s). The fluid height (H) different hydraulic columns of the microfluidic platform may be different to establish different flow rates under different hydraulic pressures. Typically, the depths of the chambers 16a-16e range between about 10 μm and about 4000 μm at the chamber openings 12a-12e.

FIG. 2D is a diagram of the microfluidic platform 90. The assembly of the top layer 40 and the bottom layer 50 form the microfluidic platform 90 with chambers 46a-46c. Each of the chambers 46a-46c includes opening pairs 42a-42c positioned over the gel channels chambers 44a-44c. The gel channels 44a-44c typically have a height (h) between about 10 μm and about 2000 μm.

Hydraulic columns 92 may be connected to the one or more chambers 46a-46c to provide flow through the chamber(s). The fluid height (H) in different hydraulic columns of the microfluidic platform may be different to establish different flow rates under different hydraulic pressures. Typically, the depths of the chambers 46a-46c range between about 10 μm and about 4000 μm at the chamber openings 42a-42c.

FIG. 2E is a diagram showing another embodiment of the microfluidic platform 90 where the one or more chambers 46a-46c are interconnected with tubing 94, which is in turn connected to a pump via connection 96, to establish perfusion through multiple tissue systems.

Single Channel Microfluidic Devices Using Insertion

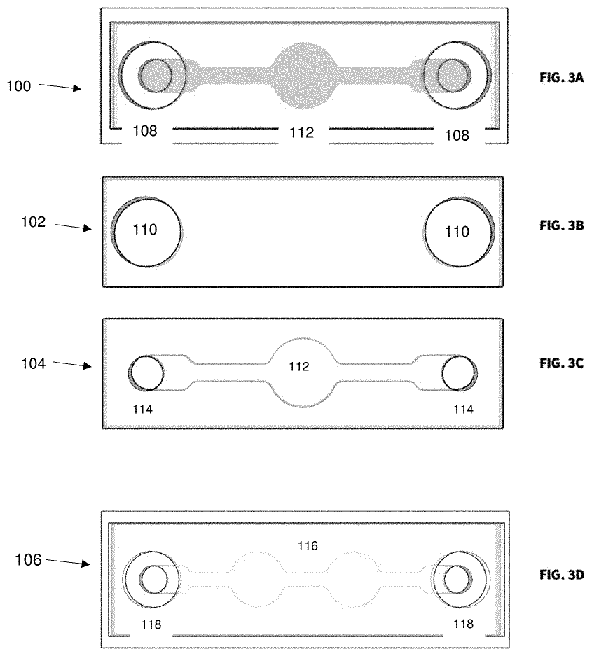

FIG. 3A is a prospective view of a single channel device 100 used for insertion of gel solution and/or cells/tissue through the gel loading port 108, without a top port in the insertion region. The device 100 consists of two layers 102, 104. The top layer 102 includes a plurality of open ports 110, with each opening pair includes two openings, at the gel loading port (FIG. 3B). The bottom layer 104 includes a plurality of single gel channels 112 with a pair of openings 110 at the gel loading port 114 (FIG. 3C). Openings 110 in the top layer 102 are usually larger, or at least the same size as the corresponding openings 114 for the bottom layer 104. Multiple insertion regions can be connected in series by tissue zones. As shown in FIG. 3D, in one embodiment 106, a single channel 116 consists two insertion zones 118 that are separated by two or three tissue zones. This can be achieved either using an ‘open-top’ design, or a device without an opening port, wheret tissue is inserted through a gel loading port.

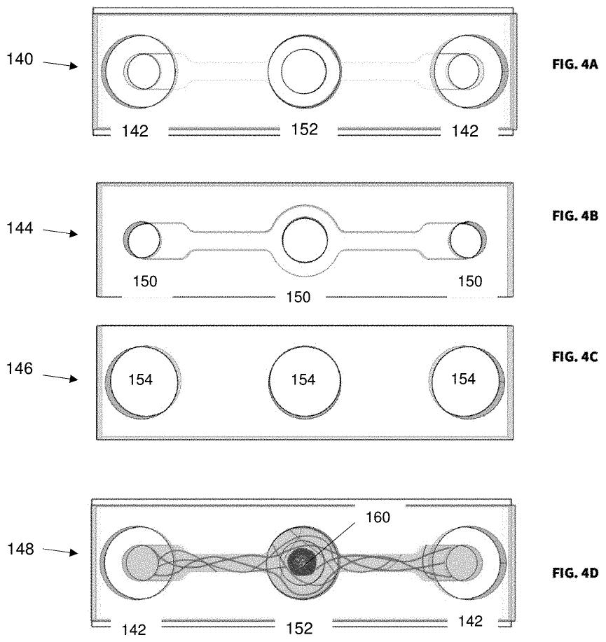

FIG. 4A is schematics of a single channel device 140 for insertion through the top port 142. The top layer 144 includes a plurality of three openings 150, with openings at the gel loading ports 142 and the insertion region 152 (FIG. 4B). The bottom layer 146 includes a plurality of three openings 154, with openings at the gel loading ports 142 and the center insertion region 152 (FIG. 4C). The tissue masses, such as organoids, tumors and spheroids, can be inserted through the top opening in the insertion region 152 right after seeding of the gel containing ECs and supporting cells, or after the perfusable microvasculature 160 are fully formed in the device 148, as shown in FIG. 4D.

### III. Methods of Making the Microfluidic Platforms

The microfluidic platform is typically formed of polydimethylsiloxane (“PDMS”), polysulfone (“PSF”), and other materials that are biocompatible, easily molded or shaped, and preferably inert. PDMS is a versatile elastomer that is easy to mold, and PSF is a rigid, amber colored, machinable thermoplastic. Other suitable materials include biologically stable thermosetting polymers, including polyethylene, polymethylmethacrylate, polyurethane, polysulfone, polyetherimide, polyimide, ultra-high molecular weight polyethylene (“UHMWPE”), cross-linked UHMWPE and members of the polyaryletherketone (“PAEK”) family, including polyetheretherketone (“PEEK”), carbon-reinforced PEEK, polyetherketoneketone (“PEKK”), cyclic olefin polymers or copolymers (“COP: or “COC”). Preferred thermosetting polymers include, but are not limited to, polyetherketoneketone (“PEKK”) and polyetheretherketone (“PEEK”).

Methods of making the platforms include stereolithography, soft lithography, laser machining, laser cutting, micromachining, micromilling, curing, bonding, three-dimensional printing, injection molding, micromolding, and setting.

In some embodiments, the top layer and the bottom layer are made separately and then bonded together. In other embodiments, the top layer and the bottom layer are formed together through additive manufacturing.

The platform may be sized to a cover glass to permit easy microscopic evaluation of tissues in the gel channel. The platform may be formed of any size suitable to accommodate a desired number of chambers. The platform typically includes between about 2 and 12 chambers, between about 2 and 10 chambers, between about 2 and 6 chambers, 5, chambers, 4, chambers, 3 chambers, or 2 chambers. The platform may include a single chamber. Suitable sizes for the platform sides are between 5 mm and 280 mm. For example, the platform may have its sides with a length and width between about 5 mm and 280 mm, between about 10 mm and 200 mm, between about 10 mm and 100 mm, between about 20 mm and 60 mm, between about 30 mm and 50 mm. In some embodiments, the platforms are glass cover sized and are about 40 mm in length and 24 mm in width.

### IV. Methods of Using the Microfluidic Platforms

The platforms are useful for forming in vitro perfusable vascular tissues and perfusable vascular tissue masses.

Typically, the methods include filling the gel channel with a gel forming solution, seeding the gel channel of the microfluidic platforms with a mix of cells and one or more extracellular matrix (“ECM”) components, and then flowing medium through the gel channel. Typically, the cells are added to the gel solution which is then inserted into the channel where it polymerized.

A. Establishing In Vitro Perfusable Vascular Tissues and Perfusable Vascular Tissue Masses

1. Forming Vascularized Tissue

The microfluidic platforms are used to form and culture perfusable vascularized tissues and/or perfusable vascularized tissue masses. The cells and ECM forming tissues and/or tissue masses are typically mixed together and deposited in the one or more tissue zones of the gel channel of the chamber. A fluid flow through the gel channel is established through differences in hydraulic pressure of the fluid passing through the channel. After culturing the cell and ECM mix for a period between about 2 days and 10 days, vascular networks develop in the trapping zone or insertion region between the two tissue zones and throughout the gel channel. If tissue masses form in the tissue zones of the gel chamber, the tissue masses are prevented from directly contacting one another, but communicate through the vascular networks. Typically, the perfusable vascularized tissues and/or perfusable vascularized tissue masses in the tissue zones of the gel channel are connected indirectly, via the vascular network of the trapping portion or insertion region.

The mix of cells and one or more ECM components are typically seeded in the one or more of the tissue zones of the gel channel. The mix of cells and one or more ECM components seeded in at least two tissue zones of the gel channel may be the same mix of cells and ECM components or a different mix of cells and ECM components. The cells typically include cells forming vasculature, such as endothelial cells, stromal cells, smooth muscle cells, pericytes, fibroblasts, progenitor cells, and combinations thereof. The cells may be transformed cells. The ECM components typically include proteins and glycosylated proteins, such as fibrous proteins (collagen, fibrin, fibronectin, elastins, and laminin), hyaluronic acid, and proteoglycans.

The mix may include additional cells for forming tissue masses, such as tumor cells, progenitor cells, patient-specific primary cells, pluripotent cells embryonic cells, and combinations thereof.

The step of flowing medium through the gel channel typically includes flow of culture medium through the channel controlled by hydraulic pressure applied to a culture medium.

Generally, the cells and one or more extracellular matrix components are mixed in the gel chamber for a period between about 2 and about 10 days. Perfusable vascular tissues and perfusable vascular tissue masses typically form after about 2, 3, 4, 5, 6, 7, 8, 9, or about 10 days in in vitro culture.

It is important to include an appropriate gel-liquid interface for successful perfusion. This can be achieved using methods which remove gel at the seeding inlet and outlet, thereby allowing for a gel-liquid interface to form.

The gel chamber may also include one or more ports for access to the tissue being cultured, or media used therein, for example, for analytical purposes or fluorescent measurements.

Top ports may also be used to seed additional cells, for example, tumor spheroids or organoids on top of the gel.

The gel channel can be of varied with, for example, to mimic an arteriole-capillary-venule arrangement with different vessel dimensions. Larger diameter vessels in the wider channel upstream of the device serve as arteriole, small diameter vessels in the center of the device serve as capillary, and larger diameter vessels in the wider channel at downstream of the device serve as venule.

A multichannel derivative device based on single channel device can also be used to model blood-lymphatic systems. For example, a parallel gel channel with partial walls adjacent to the main single channel device is constructed. The other side of gel channel is connected to a media channel.

A single channel device is seeded with blood vascular cells to form blood vessels through vasculogenesis. Lymphatic endothelial cells can be seeded as a monolayer on the parallel gel surface to engineer lymphatic vessels by lymph-angiogenesis. This arteriole-capillary-venule construct can be further connected with the lymphatics that the lymphatic channel connecting with the downstream end of the ‘venules’.

Skin models can also be constructed using the same devices, by selecting appropriate cells for skin and blood vessels.

The gel channel can also contain structures, for example, flexible PDMS pillars located at the center of a single channel device, with a distance about 5 mm. Muscle cells (such as skeleton muscle, cardio muscle) can be seeded with collagen into this two-pillar single channel device. A muscle bundle will form attached to the pillars. After muscle bundle formation, vascular cells can be seeded around the muscle bundle to vascularize muscle in single channel device. The deformation of these 2 pillars can be used for muscle force measurements.

2. Using IF to Control Morphology and Perfusability

As demonstrated by the examples, it was discovered that culturing cells under static conditions can be used to form vascularized tissues. However, as further demonstrated by the examples, IF is a critical factor in controlling the morphology and perfusability of the tissue masses.

MVN formed under IF exhibited improved morphologies compared with MVN grown under static conditions with higher percent coverage area, larger vessel diameters and greater network perfusability. Moreover, MVN morphologies are determined by global IF rather than local IF.

IF is applied either by gravity using a head pressure and/or a pump. Conditions should be within the same ranges as interstitial fluid flows in normal tissue.

3. Inhibiting the MMP-2 Pathway

It was discovered in the course of conducting the studies described below that the MMP-2 pathway plays a major role in the morphology and perfusability of developing vascular tissue. IF treatment improves vasculogenesis capacity of ECs through upregulation of matrix metalloproteinase-2 (“MMP-2”). As a result, the interplay between IF and MMP-2 inhibitor can be used to regulate key morphological parameters of the self-organized MVN, with perfusability well maintained. The boosted vasculogenesis capacity of ECs which results from upregulated MMP-2 when cultured under IF provides a means to regulate many important morphological parameters of self-organized MVN, with perfusability and permeability well maintained.

B. Interconnected tissue masses

Methods of forming and culturing perfusable vascularized tissue masses interconnected solely via vascularized networks have been developed.

The majority of current tumor spheroid in vitro models lack perfusable vascularization. Only a few types of tumor cells form limited vascularization in terms of vascular coverage, density of penetrated microvessels, as well as the perfusability of the microvessels. These models typically do not recapitulate the vasculature-rich tumor microenvironments and cannot be used for tumor-associated vasculature studies, such as immune cell recruitments and tumor cell extravasation.

The microfluidic platforms permit vascularization of tumor spheroids by introducing flow and culturing endothelial cells, stromal cells, and tumor cells in a confined structure. The vascularized tumor spheroids mimic the tumor microenvironments in vivo. The improvement of vascularization in tumor spheroids prolongs the life span of both tumor spheroids and the vasculature, which makes it possible to perform long-term studies. Integrating patient-derived organotypic tumor spheroids (“PDOTS”) makes this platform a better system to evaluate the efficiency of pre-clinical treatments, such as immune checkpoint blockade or chemotherapy.

1. Body-on-Chip Model

The microfluidic platforms improve the vascularization of organoids, and connect the vasculature within the organoids to the capillary bed around the organoid, to form a perfusable vascularized organoid. Unlike the artificial endothelium, the microvascular networks emerge from endothelial cells and stromal cells, which recapitulate the key features of capillary beds in vivo, such as permeability. The perfused vascular networks in organoids prolong their lifespan and reduce the necrotic core. The microvascular networks in each channel can be made from organ specific endothelial cells and stromal cells to match the organoid in the same channel. The individual perfusable organoid channels can be further connected to make a body-on-chip model. Since the vasculatures in each channel are made of organ-specific endothelial cells and stromal cells, this body-on-chip model better mimics the in vivo scenario that the vasculature in different organs has distinct features.

Studies demonstrate tissue with endothelial cells forming a perfusable vascular network. These vascular networks typically form after 5-7 days of culture in the microfluidic platform. Tissue formed with tumor surrounded by the tumor associated fibroblasts.

2. Metastasis-on-Chip Model

A tumor metastasis model was also established by combining the tumor spheroids and organoid models, with cells from the primary tumor spheroids intravasating to surrounding vasculature, migrating along the microvessels under the flow, extravasating, and seeding metastasis in organoids, such as liver, lung, or brain organoids. This has not been achieved by any current in vitro models. Many studies can be performed using these models, such as drug or immune checkpoint blockade treatments and immune cell perfusions.

3. Blood Brain Barrier (“BBB”) Model

As demonstrated in the examples, the system can also be used to create a model for the blood brain barrier by culturing endothelial cells, astrocytes and pericytes. As the control, BECs, pericytes and astrocytes were cultured in static condition. At day 6, BECs self-organized into MVN with extremely skinny vessels. Those vessels seemed lack of continuous patent lumens, which was evidenced by failed dextran perfusion. Cells were then seeded triculture in single channel devices grew under IF. BECs developed into fully perfusable MVN and are associated excellent barrier function. IF drastically altered morphology of BBB MVN, significantly increased diameters of perfusable vessels, and substantially boosted the perfusability of formed MVN.

C. Accurate Vascular Permeability Measurement

Vascular permeability is a crucial factor regarding the functionality of engineered microvasculature. The single channel device is ideal for vascular permeability measurement. The long gel region eliminates contamination from free diffusion of dextran dye through porous hydrogel, which is an issue for the typical three channel design and some commercial MVN chips. Also because of the large gel region and the application of IF, characteristic length scale of the perfusable MVN formed is in the order of centimeters, one order of magnitude larger than typical self-organized MVNs formed statically. This provides valuable insight regarding engineering vascularized bulk tissues through self-organizing mechanism, instead of the commonly used pre-patterning techniques.

The present invention will be further understood by reference to the following non-limiting examples.

The examples demonstrate fluidic platforms and IF can be used to:

1) form perfusable microvascular networks,

2) form perfusable vascularized single or multiple tumor spheroids made of tumor cell lines or derived from patients,

3) form perfusable vascularized single or multiple organoids,

4) the combination of 3) and 4) to establish organ specific metastatic models,

5) perfuse drugs or other types of cell to vascularized tumor spheroids or organoids.

6) accurate measurement of vascular permeability

The examples also demonstrate the significance and how to manipulate interstitial fluid flow to modulate formation of organoids and tumor spheroid growth in the microfluidic platforms.

The platforms provide a means to create vascularized tumor spheroids and organoids, and provide perfusable vascular connections that better mimic physiological or pathological microenvironments for organoid and tumor studies. The improved organoid/spheroid vascularization in combination with the perfusable vascular networks reduces the necrotic core, prolongs the lifespan of the organoids/spheroids, and perfuses drugs or other types of cells to tumor spheroids or organoids, making it possible to study cell dynamics between multiple organoids/spheroids, for example, metastasis from a tumor spheroid to organoids interconnected through vascular networks.

### EXAMPLE 1

### Microfluidic Platform Design, Manufacture, and Assembly Formation of Perfusable Vascularized Tumor Spheroid Model

Materials and Methods

The design of the fluidic platform is used to create perfusable vascular networks connecting vascularized tumor spheroids and organoids. This fluidic platform contains two polydimethylsiloxane (“PDMS”) layers on top of cover glasses: the top layer serves as medium channel reservoirs and connections; the bottom layer has spheroid or organoid trapping gel channels (FIGS. 1A-1C). The dimensions of the device are shown in FIG. 1A-1C.

Both top layer and bottom layer platforms were manufactured by laser-cutting or micro-milling of PDMS. The cured PDMS was punched with 4 mm (top layer) or 2 mm (bottom layer) diameter punches to open the medium channel connections and gel seeding ports. These two layers were treated with plasma to bond them together, and then the cover glass bonded to the bottom platform.

After assembling the fluidic devices, endothelial cells (“ECs”) and stromal cells were mixed in solution containing extracellular matrix (“ECM”, such as fibrin) and seeded into the gel channels. Interstitial flow was applied at different hydraulic pressures by placing culture medium at different heights (32 in FIG. 2C) to optimize the flow condition (FIGS. 2A-2C). Vascular networks formed after 5-7 days.

A single tumor spheroid along with ECs and stromal cells mixed together with ECM was similarly seeded into the gel channel. Interstitial flow was applied using hydraulic pressure, as described above.

Results

The maturation time of vascularization in the tumor spheroid differed by cell types. A perfusable vascularized tumor spheroid formed 5 days post-seeding.

This demonstrated that the platforms can be used to form and culture vascularized tumor spheroids, vascularized organoid, perfusion through vascularized tumor spheroids, and perfusion through vascularized organoid. The results also demonstrate that the platforms can be used to establish multiple perfused spheroids/organoid models, and organ-specific metastasis models.

### EXAMPLE 2

### Interstitial Flow Promotes the Formation of Functional Microvascular Network In Vitro Through Upregulation of Matrix Metalloproteinase-2

Self-organized microvascular networks (MVN) have become key to the development of many microphysiological models and a valuable tool for diverse applications in tissue engineering. However, the self-organizing nature of this process combined with variations between types or batches of endothelial cells (ECs) in their ability to form functional vessels often lead to inconsistency or failure to form a functional MVN. Since interstitial flow (IF) has been reported to play a beneficial role in angiogenesis, vasculogenesis and 3D capillary morphogenesis, the role IF plays during neovessel formation was studied in a customized single channel microfluidic chip for which IF has been fully characterized.

Materials and Methods

### Analytical Solution of Interstitial Flow Across Fibrin Gel

Matrix permeability of the fibrin gel was measured by the displacement of media in syringe reservoirs. The Darcy permeability (K) of acellular fibrin gel was calculated by Darcy's law:

$$\frac{Q ( t )}{A} = \frac{K Δ P ( t )}{μ L}$$

where Q is the volumetric flow rate, A is average cross section surface area of gel, μ is the viscosity, ΔP is the pressure difference across the gel, and L is the length of gel in the direction of interstitial flow.

The pressure difference in the syringe reservoirs was determined by the difference in the height of liquid Δz, assuming identical syringes were used in inlet and outlet:

$$\begin{matrix}Δ P ( t ) = ρℊΔ z ( t ) \\ Δ z ( t ) = Δ {z}_{0} - \frac{1}{{A}_{r}} \int Q ( t ) dt - \frac{1}{{A}_{r}} \int Q ( t ) dt\end{matrix}$$

where Δz₀ is the initial difference in liquid levels, Aᵣ is the cross section area of syringe, ρ is density of media, g is gravity acceleration.
Equation (1)-(3) give

$$Q ( t ) = \frac{AK ρℊΔ {z}_{0}}{μ L} {e}^{- \frac{2 AK ρℊ}{μ {LA}_{r}}}$$

thus V(t), decreased liquid volume in the first syringe, which equals the increased volume in the second syringe, is calculated as

$$V ( t ) = \int Q ( t ) dt = \frac{1}{2} Δ {z}_{0} {A}_{r} ( 1 - {e}^{- \frac{2 AK ρℊ}{μ {LA}_{r}} t} )$$

By transforming equation (5), The Darcy permeability (K) is

$$K = - \frac{μ {LA}_{r}}{2 ρℊ At} log ( 1 - \frac{2 V ( t )}{Δ {z}_{0} {A}_{r}} )$$

Based on this calculation and the initial measurements, the Darcy permeability (K) of acellular fibrin gel (fibrinogen 3 mg/ml) used in this experiment is K=1.2×10¹³;

Single Channel Device Design

The initial goal was to design a single channel microfluidic device that could maintain relatively stable interstitial flow for 24 hours, which is the time interval of replenishing culture media. Based on the analytical solution and measured Darcy permeability, the device was designed with gel region length L=15 mm, height H=0.5 mm, and width W=2 mm. With those parameters, the time evolution of liquid volume in syringe reservoirs are plotted, and the ratio of pressure difference at t=24 h and t=0 is

$$\frac{Δ P ( t = 24 h )}{Δ P ( t = 0 )} = 74.55 \%$$

which means a relatively stable interstitial flow can be maintained in such devices.

To investigate the vessel formation under IF, two types of microfluidic devices were fabricated. A common three channel device was used to culture MVN statically, as shown in FIG. 3A. Another device with a single rectangular gel region was used to culture MVN under IF (FIG. 3B). Height difference of culture medium in the reservoirs hooked at inlet and outlet drives IF through the whole gel region. Through Darcy's law, it was possible to measure matrix permeability from liquid volume accumulated in the outlet reservoir. With the measured matrix permeability of acellular fibrin gel, dimensions of the microfluidic device were chosen to make sure IF can be maintained for 24 hours, when culture medium was replenished to restore the pressure gradient. The relative stable IF speed was confirmed with

$$\frac{{v}_{IF} ( t = 24 h )}{{v}_{IF} ( t = 0 )} = 74.6 \%$$

using analytical solutions and measured matrix permeability.

To ensure that fibrin gels are stable and all the differences observed in MVNs resulted from cells embedded in the hydrogel, 10, 20 and 30 mm H₂O pressure differences were applied to acellular fibrin gel seeded in device for 6 days. Average IF speeds were calculated from the volume of accumulated culture medium in outlet every day.

Next, HUVEC with fibroblast were seeded into microfluidic devices to form MVN. IF was induced with 10, 20, or 30 mm height differences of culture medium between inlet and outlet.

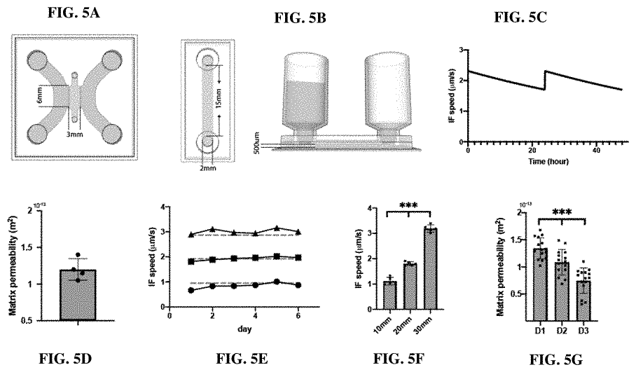

To systematically investigate the effect of IF on vasculogenesis in vitro, HUVECs and HLFs were co-cultured in fibrin gel under static and various IF conditions. For static condition, the microfluidic device with a central gel region with two media channels flanked along each side, shown in FIG. 5A, which is widely utilized to produce in vitro MVN. IF experiments were performed using single channel devices as described herein and shown in FIGS. 1, 2, 3, 4, and 5B, as well as a three channel device (two culture media separated by a single gel channel). Fibrin gels were subjected to 10, 20, or 30 mm H₂O pressure difference to produce different levels of IF.

Green fluorescent protein (“GFP”) tagged human umbilical vein endothelial cells (HUVECs) and human lung fibroblasts (HLFs) were seeded in a 7:1 ratio for all static and IF experiments. Devices were imaged under confocal microscopy with dextran dye perfused at day 6, when MVN were interconnected and perfusable. Devices were seeded with HUVECs and HLFs in combination and HUVECs alone.

Morphology and perfusability were measured, as was expression of tissue plasminogen activator (tPA) and urokinase (uPA), both involved in the plasmin pathway, as well as MMP-2. To reveal the underlying mechanism, quantitative PCR was performed on HUVECs extracted from the 3D gel at day 3 during neo-vessel formation cultured under both static and IF conditions to evaluate gene levels. A series of protease inhibitory experiments were conducted to identify the specific enzymes that are responsible for the significant differences induced by IF treatment. As fibrin was used to form the 3D gel scaffold in this study, initial focus was on plasminogen activator (PA)-plasmin system because it is the most typical path for fibrinolysis.

Results

In Vitro Microvascular Network Formation Under Interstitial Flow

Acellular fibrin gel is stable under the experimental setup for at least 6 days (FIG. 5E). Furthermore, the measured IF speeds agree well with the analytical calculation.

In the first 24 hours, different pressure gradients lead to distinct IF speeds. The measured average IF speeds are similar to the IF speeds in acellular fibrin gel (FIG. 5F), which means the embedded cells do not alter the matrix permeability initially. However, as the cells remodel ECM and self-organize into MVN, the effective matrix permeability drastically changes. See FIG. 5G. The effective matrix permeability keeps decreasing for the first 3 days, possibly due to secreted ECM by FBs and ECs. After that, as the nascent vessels progressively connected with each other and the MVN become gradually perfusable, much faster luminal flow takes over IF and equilibrated the restored pressure difference in only a few hours. At day 6, ECs self-organized into densely connected 3D MVN mimicking morphology of in vivo capillaries. Fully perfusable MVN across the whole gel region over the length of 15 mm were confirmed by perfusing dextran dye from inlet.

Interstitial Flow Boosted the Formation of Functional MVN

Clear morphological differences can be observed in projected images acquired for devices cultured under static and various IF conditions. To better quantify those differences, morphological parameters were measured from projected images. The quantification clearly showed that vessel morphology was noticeably affected by IF treatment. MVN formed under IF covered significantly more area with perfusable vessels. The largest perfusable vessel coverage occurred when subjecting the cells to low IF, and kept decreasing as higher IF was implemented. The number of branch points and average branch length were significantly affected by IF treatment. Fewer branch points and longer branch length were observed as IF increased. Vessel diameter is another parameter that is strongly affected by IF. In comparison to MVN formed statically, IF treatment led to MVN with significantly larger diameters of perfusable vessels. Stronger IF engendered MVN with significantly larger vessel diameters compared to weaker IF.

Perfusability is a crucial factor when evaluating the quality of engineered MVN, since the major function of vessels is to transport nutrients, oxygen, metabolic wastes and various cells through perfusable lumens. Perfusability is measured as the ratio between dextran dye cover area (perfusable vessels) and GFP HUVEC cover area (all the vessels). Usually vessels with larger diameter are easier to perfuse, so MVN treated with higher pressure IF should exhibit an overall higher perfusability. To better interpret perfusability, MVN with at least one perfusable vessels across the gel region is defined as partially perfusable, and MVN with perfusability over 80% is defined as fully perfusable.

When cultured under static condition, only 50% of MVN were fully perfusable and 77% were at least partially perfusable. IF substantially boosted perfusability of MVN, with all of them fully perfusable under various IF conditions. Forming perfusable vessels in a single channel device is more challenging because patent lumens must be connected all across the gel region over 15 mm, while perfusable vessels only need to be connected over the width of 3 mm across gel region when cultured statically in the 3-channel device.

In the single channel device, nutrients are gradually consumed by cells as the IF travels along the gel. If the gel region is excessively long, at one point it will fail to support the formation of functional MVN at downstream due to depletion of nutrients. To make sure MVNs formed in the single channel device were not affected by this limitation, the morphology at the inlet, middle, and outlet region were quantified separately under various IF conditions. It was found that similar vessel diameters in different regions when cultured under certain IF ensured that MVNs were formed with similar morphology all over the gel region under the experimental setup.

Overall, MVN formed under IF exhibited distinct morphologies with larger coverage area and vessel diameters, compared with MVN grown statically. Furthermore, the variances seemed to be greatly reduced for all morphological parameters quantified in IF cases, indicating IF regulates the in vitro neovessel formation in a robust and replicable manner. Moreover, IF treatment significantly boosted the perfusability of self-organized MVN, leading to a robust way of forming fully perfusable MVN in vitro.

IF Affects Global MVN Structures Within Microfluidic Device

To better understand the role IF plays during neovessel formation, a single channel device with large circular middle region and narrow neck regions was designed. The same setup of reservoirs was used to maintain pressure gradient along the hydrogel. This allowed generation of inhomogeneous IF within the gel. Matrix permeability was used as the value to measure acellular fibrin gel in this study. The distribution of IF over the whole device was calculated in COMSOL. Four regions with the same area size were chosen as regions of interest (ROI). Average IF speed in those ROIs were calculated under either 10 mm (100 Pa) or 20 mm (200 Pa) H₂O pressure difference.

The relation of head height on speed is shown in FIG. 7B.

IF speeds in neck ROIs were about three times stronger than the speed in middle ROIs. However, further quantification did not reveal any clear differences in perfusable vessel diameters, coverage area and number of branches between neck and middle ROIs within devices under the same pressure difference, suggesting MVNs were formed with relative uniform morphology. Distinct MVN morphologies between 100 Pa and 200 Pa pressure difference were present throughout, further demonstrating that MVNs formed under stronger IF were associated with larger vessels, smaller coverage area and less branches.

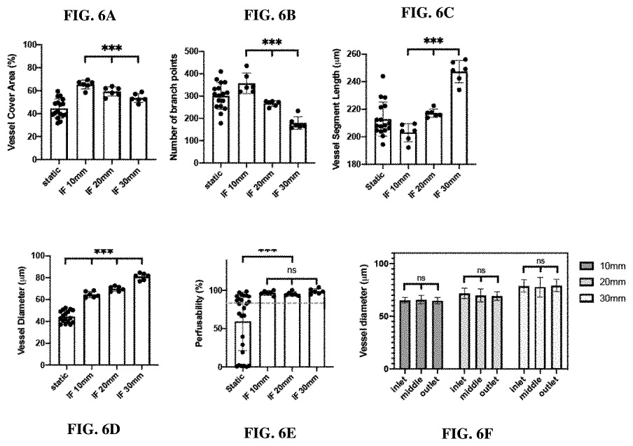

The effect of local IF on local morphology was further examined, especially comparing MVN morphologies between the neck region of devices under 100 Pa pressure difference, and the middle region of devices under 200 Pa pressure difference. Results are shown in FIGS. 7A-7F. As a consequence of the non-uniform gel region, IF speed in the former region, although under a smaller global pressure difference, was actually almost twice the IF speed in the latter region. Results are shown in FIG. 6A-6F. MVN in the neck region subjected to 100 Pa global pressure (higher local IF speed) were associated with narrower vessels, larger coverage area and more branch points compared to the MVN in the middle region subjecting to 200 Pa global pressure (lower local IF speed), which is opposite with what was previously discovered. Those findings essentially suggest that MVN morphologies are determined by global IF rather than local IF. It is very likely that those HUVECs produced secretomes, whose production are altered by IF and are crucial for neovessel formation, work globally as they are transported by IF and diffusion. This is particularly important for microfluidic design planning to incorporate IF to boost the MVN formation. Even aiming for engineering MVN with uniform morphology, there is no need to put particular considerations in the design to accommodate for homogenous distribution of IF.

### EXAMPLE 3

### Role of Plasminogen and MMP-2 in Neovessel Formation

In order to self-organize into functional MVN, ECs must intensively remodel the surrounding ECM, mostly through cleavage of ECM components by secreted enzymes from embedded cells. Since larger vessels in MVN consistently formed under IF, it was hypothesized that IF elevates the production of proteases, which enhances the vasculogenesis capacity of ECs. In the systems, however, HLFs were also seeded as supporting cells with HUVECs, thus making it hard to decouple the effect of IF on each type of cells.

The role of MMP-2 was also examined.

Materials and Methods

HUVECs were seeded alone in the three channel devices shown in FIG. 3A and cultured statically.

HUVEC monoculture of the same concentration were also seeded in single channel devices of FIG. 5B and subjected to IF.

The expression levels of a few genes related to plasminogen, including PLAT, PLAU, PLAUR, SERPINE1, ANXA2, S100A10, THBD, were quantified.

Culture media was also supplemented with high concentration (100 uM) plasminogen activator inhibitor-1, which inhibits both uPA and tPA, or aprotinin, which inhibits plasmin.

To confirm if MMP affects neo-vessel formation in fibrin scaffolds, HUVEC were grown in monoculture under IF in the microfluidic device with 50 μM marimast, which is a highly potent broad spectrum MMP inhibitor.

To identify specific MMPs that are crucial for vessel formation in the system, quantitative PCR were performed on a series of MMPs from HUVECs cultured in 3D fibrin gel under static or IF conditions,

ARP-100 (IC₅₀ values are 12, 200, 4500, >50000 and >50000 nM for MMP-2, -9, -3, -1, -7, respectively) and NSC 405020 (selective inhibitor of the collagenolytic activity of MT1-MMP without affecting the catalytic activity of cellular MT1-MMP) were tested as specific inhibitors for MMP-2 and MMP-14. 14. High concentrations of anti-MMP-1 neutralizing antibody were used to inhibit MMP-1 activity

Results

### IF Enhanced the Vasculogenesis Ability of EC Through Upreguation of MMP-2

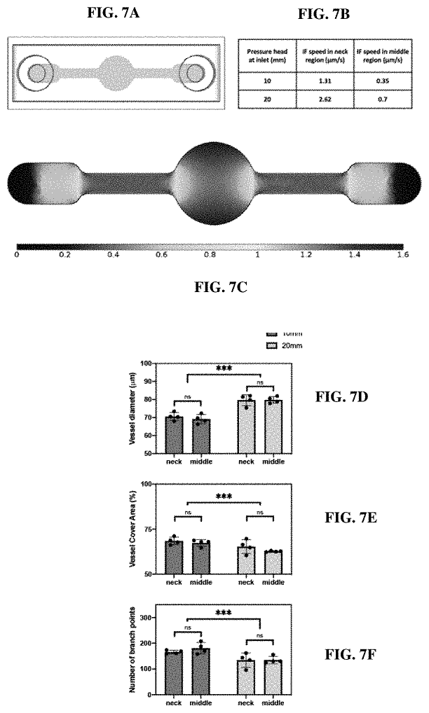

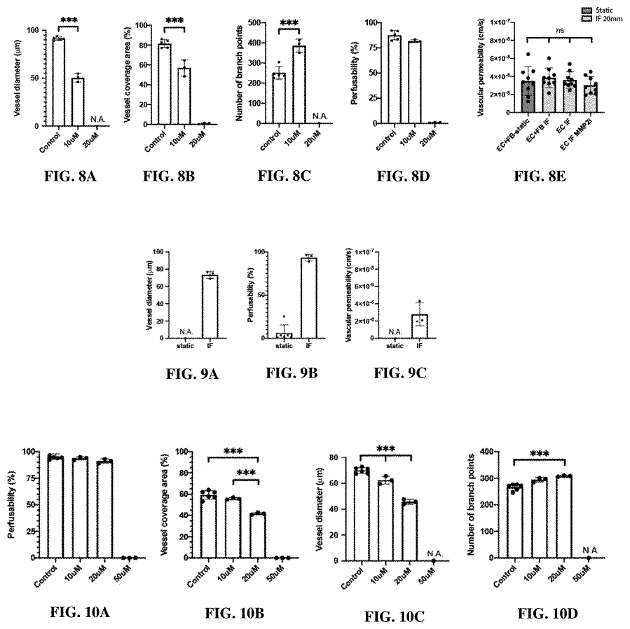

Results for using HUVEC monoculture are shown in FIGS. 8A-8D.

Results for HUVEC and HLF coculture are show in FIGS. 10A-10D.

Vascular permeability is shown in FIG. 8E.

No perfusable vessels were found for HUVECs seeded alone and cultured without IF. Even though ECs seemed to form a connected network, they were not able to make continuous patent lumens. However, HUVEC monoculture of the same concentration seeded in single channel devices subjected to IF exhibited tremendous improvement in neo-vessel development. IF drastically altered the behavior of HUVECs seeded in device, and led to robust formation of perfusable fully interconnected MVN at day 5.

Tissue plasminogen activator (tPA) and urokinase (uPA) are the two agents that convert plasminogen to active plasmin, which is the protease mainly responsible for Fibrinolysis. However, no clear upregulation of the PA—plasmin system was identified after quantifying expression level of a few related genes (including PLAT, PLAU, PLAUR, SERPINE1, ANXA2, S100A10, THBD). Moreover, supplementing culture media with high concentration (100 uM) plasminogen activator inhibitor-1, which inhibits both uPA and tPA, or aprotinin, which inhibits plasmin, at 200 U/ml,did not affect the development of fully perfusable MVN by HUVEC monoculture under IF condition. Those findings clearly indicate that HUVECs remodel fibrin gel during neo-vessel formation in a plasmin-independent manner.

MMP is another common protease family consists of more than 20 members, each of which exhibits substrate specificity for numerous ECMs. Although not specifically functioning as fibrinolytic enzymes, it has been reported that a series of MMPs, including at least MMP-1, -2, -3 and MT1-MMP, have the capacity to dissolve fibrin gel. Furthermore, consistent with the finding, other groups reported endothelial cells invade fibrin ECM in a plasmin-independent manner and require the activity of MMPs. Inhibiting MMP activities completely disrupted the vessel formation process. Even after 5 days of culturing with IF, HUVECs were only able form short discontinuous segments with no signs of lumen. Endothelial cells are known to produce multiple MMPs, including MMP-1, -2, -9, -13, -14, but with variations in species and microenvironment.

To identify specific MMPs that are crucial for vessel formation in the system, quantitative PCR were performed on a series of MMPs from HUVECs cultured in 3D fibrin gel under static or IF conditions, where it was found that IF upregulated gene levels of MMP-1 and MMP-2 by 1.6 and 2.7 folds, respectively. MMP-14, also known as membrane type-1 MMP or MT1-MMP, was downregulated by IF. However, MMP-14 was still included in the subsequent inhibitory experiments because MMP-14 has been reported to be involved in the processes of EC invasion and network formation through localized basement membrane degradation.

In inhibitory tests, ARP-100 (IC50 values are 12, 200, 4500, >50000 and >50000 nM for MMP-2, -9, -3, -1, -7, respectively) and NSC 405020 (selective inhibitor of the collagenolytic activity of MT1-MMP without affecting the catalytic activity of cellular MT1-MMP) were used as specific inhibitors for MMP-2 and MMP-14. Because narrow band inhibitors with exclusive specificity for MMP-1 are not available at this moment, high concentration of anti-MMP-1 neutralizing antibody were used to inhibit its activity. The experiments showed blocking activity of MMP-1 does not affect vasculogenesis of HUVEC monoculture in fibrin gel under IF. Fully connected perfusable MVN still robustly formed with similar morphology and perfusability as in control case. Similarly, inhibiting MMP-14 didn't induce any noticeable changes in formed MVN either.

Unlike MMP-1 or MMP-14, MMP-2, which is also the most upregulated MMP in gene levels under IF, turns out to be crucial for vasculogenesis in a fibrin scaffold. MVN formation with HUVEC monoculture under IF was altered when cultured in the presence of MMP-2 specific inhibitor ARP-100 in a clear dose dependent manner. Low dosage (10 μM) ARP-100 effectively reduced the ECM remodeling capacity of HUVECs, leading to the formation of MVN with significantly narrower vessels, reduced coverage area and increased branches. When exposed to an increased concentration of 20 μM, HUVECs were only able to develop into extremely narrow network. Although the MVN seemed to be fully connected, perfusion testing confirmed no vessels can be successfully perfused with dextran dye. With even higher concentration of 50 uM, HUVECs can barely move in the 3D gel scaffold due to the strong suppression of MMP-2 activity. At day 5, most HUVECs were still not able to connect with their neighboring cells, leading to a complete failure in developing network structure. Varied concentrations of MMP-2 specific inhibitor lead to distinct behaviors of HUVECs for the whole duration of in vitro vessel formation.

In summary, MVN can be robustly formed using HUVEC monoculture grown in a 3D fibrin gel with IF treatment. Through qPCR measurement and a series of inhibitory tests, it was established that HUVEC self-organized into functional MVN in fibrin scaffold in a plasmin independent manner. It was further demonstrated that IF treatment significantly boosted vasculogenesis capacity through upregulation of MMP-2, a specific member of MMP family.

### Regulating Morphology of MVN Through Interplay Between IF and MMP-2 Inhibitor While Maintaining Functionality

MMP-2 inhibitor noticeably altered MVN formed using HUVEC monoculture under IF in a dose dependent manner. Balancing MMP-2 activity through interplay between IF and MMP-2 inhibitor can be utilized to regulate critical morphological parameters while maintaining the perfusability of engineered MVN. Using MVN developed by HUVEC monoculture under IF, the appropriate dose (10 μM ARP-100 in this case) of MMP-2 inhibitor effectively reduced the average diameter and coverage area of vessels, with the perfusability well maintained in comparison with control group. The approach was further validated using HUVEC HLF coculture, the most common cells used to engineer functional MVN in vitro. A clear dose dependent effect of MMP-2 inhibitor was observed, following the same trend as in MVN formed with HUVEC monoculture. MVN formed by HUVEC and HLF coculture can maintain high perfusability with a higher concentration (20 uM ARP-100) of MMP-2 inhibitor when cultured with IF.

In summary, MVN formed without supporting cells tends to be leakier. Since vascular permeability is a critical aspect of functionality of engineered MVN, vascular permeability of 40 kD dextran was measured and compared under various conditions where perfusable vessels can be robustly formed. This includes HUVEC and HLF co-culture under static or IF condition, HUVEC monoculture under IF, and HUVEC monoculture with 10 μM MMP-2 inhibitor under IF. No difference in vascular permeability was found across all the conditions, where MVNs exhibited distinct morphologies. Moreover, all the measurements were in the order of 10⁻⁸ cm/s, which are quite low for vascular permeability measured using 40 kDa dextran. These results indicate perfusable MVN formed under various conditions in this study are all well formed in terms of barrier function, even including the MVN formed without supporting cells. This is quite surprising since it was expected that MVN formed by HUVEC monoculture would be leakier. The excellent barrier function of MVN formed by HUVEC monoculture under IF cannot be explained alone by the findings regarding MMP-2 upregulation under IF. Gene expression levels regarding ECM secretion, cell migration, adhesion and angiogenesis on HUVECs seeded in devices cultured under static and IF conditions for 3 days. However, none of those genes were obviously altered by IF treatment.

Next, the longevity of MVN developed with HUVEC monoculture under IF was assessed. Those monoculture MVNs remain perfusable from day 5 until at least day 26. During the whole time, vascular permeabilities were well maintained with only moderate changes in morphology. It should be noted here that IF cannot be maintained after MVN become fully perfusable. Much stronger luminal flow will take over IF and equilibrate the pressure differences between inlet and outlet much faster.

Taken together, perfusable MVN formed with HUVEC monoculture under IF exhibited outstanding functionalities in terms of perfusability, permeability and longevity. Moreover, some critical morphological parameters of the self-organized MVN can be robustly regulated through interplay between IF and MMP-2 inhibitor, with functionality well maintained.

### EXAMPLE 4

### Effect of Interstitial Flow on Formation of Fully Perfusable Brain Microvascular Network

To validate that the findings and methods are generic and can be employed to boost the formation of other organotypic MVN models, the effect of IF treatment on vasculogenesis of in vitro BBB MVN was investigated.

Materials and Methods

BBB MVN were formed, consisting of primary human brain endothelial cells (BECs), brain pericytes, and astrocytes.

As the control, BECs, pericytes and astrocytes were first seeded in the 3-channel device cultured in static condition.

Triculture were then seeded into single channel devices and grown under IF.

All MVN tissues were immunstained and studied for histology and morphology. Quantitative PCR was also performed to measure MMP-2 levels. The effect of a MMP-2 specific inhibitor was also tested in the BBB model to antagonize the effect induced by IF, as observed in MVNs formed with HUVEC monoculture or HUVEC and HLF co-culture.

Results

Results are shown in FIGS. 9A-9C.

At day 6, BECs self-organized into MVN with extremely skinny vessels. Those vessels lacked continuous patent lumens, which was evidenced by failed dextran perfusion. This morphology is similar to the morphology of the MVN formed by HUVEC monoculture in static conditions or under IF with suppression of MMP-2 activities.

BECs developed into fully perfusable MVN and are associated excellent barrier function. IF drastically altered morphology of BBB MVN, significantly increased diameters of perfusable vessels, and substantially boosted the perfusability of formed MVN, comparable to the results with vascularized tissue in Example 2. When cultured in static conditions, perfusable vessels were only rarely observed and most MVN were not perfusable at all.

In comparison, all of the MVNs treated with IF were fully perfusable, further endorsing the effectiveness of IF in forming functional MVN in vitro. Immunostaining revealed PCs and ACs reside in interstitial space surrounding microvessels formed by BECs in both static and IF conditions. PCs and ACs were in direct contact with endothelium, similar to other BBB models. However, no clear differences were observed in distributions and morphologies of ACs and PCs in static or IF condition, suggesting the substantial changes in MVN grown under IF most likely resulted from BECs.

Quantitative PCR was further performed on BECs sorted out from the triculture within fibrin gel at day 3 during neovessel formation to evaluate gene levels of MMP-2. mRNA of MMP-2 was increased (1.7±0.26 fold) under IF, which is consistent with observations in MVN formed with HUVEC, indicating IF triggers the same downstream effect in both cases among different types of ECs.

The effect of an MMP-2 specific inhibitor was also tested in the BBB model to antagonize the effect induced by IF, as observed in MVNs formed with HUVEC monoculture or HUVEC and HLF co-culture. Differences were observed during BBB network formation under IF. On day 3, higher concentrations of MMP-2 inhibitor (20 uM) lead to thinner and sparser vessels compared with the ones treated with lower concentration (10 μM). At day 6, BBB network with low concentration of MMP-2 inhibitor became perfusable with much thinner vessels compared to untreated ones. In contrast, networks cultured with higher concentration of MMP-2 inhibitor exhibits disconnected thin network segments without any perfusable vessels.

Unless defined otherwise, all technical and scientific terms used herein have the same meanings as commonly understood by one of skill in the art to which the disclosed invention belongs. Publications cited herein and the materials for which they are cited are specifically incorporated by reference.

Those skilled in the art will recognize, or be able to ascertain using no more than routine experimentation, many equivalents to the specific embodiments of the invention described herein. Such equivalents are intended to be encompassed by the following claims.

We claim:

1. A microfluidic platform for a perfusable tissue culture, the platform comprising
a chamber comprising chamber openings overlaid a gel-filled channel,
the gel channel comprising at least two contiguous, and aligned tissue zones and another zone positioned between the at least two tissue zones used to trap or insert an organoid, spheroid or tumor sample,
wherein the gel channel optionally includes one or more ports for media collection and/or analysis,
the chamber opening having connections to reservoirs or a pump for interstitial fluid flow, and preferably including a gel-liquid interface.

2. The microfluidic platform of claim 1, wherein the at least two tissue zones, at their widest, have a width at least two times greater than the width of the trapping portion.

3. The microfluidic platform of claim 1, wherein the gel channel has a length between about 2 times and 50 times greater than the length of the trapping portion.

4. The microfluidic platform of claim 1, wherein the tissue zones have a width between about two times and 30 times greater than the width of the trapping portion, or wherein the gel channel has a variable width.

5. The microfluidic platform of claim 1, comprising one or more reservoirs for media or tubing for a pump connected to the chamber.

6. The microfluidic platform of claim 1, wherein the chamber comprises two chamber openings and the gel channel comprises two tissue zones.

7. The microfluidic platform of claim 1, wherein each of the tissue zones contacts one chamber opening.

8. The microfluidic platform of claim 1, wherein the chamber comprises two openings and one gel channel comprising two tissue zones, a portion of each of the two tissue zones connected to one of the chamber openings.

9. The microfluidic platform of claim 1, wherein the platform includes a plurality of chambers.

10. The microfluidic platform of claim 1, wherein cells or tissue can be inserted into the device either on top of or into the gel region, preferably either on top through the top opening with the ‘open-top’ device, or inserted through the gel loading port.

11. The microfluidic platform of claim 1, wherein the gel channel comprises one or more extracellular matrix components.

12. The microfluidic platform of claim 11, wherein the gel channel comprises fibrin.

13. The microfluidic platform of claim 1, wherein the gel channel comprises endothelial cells.

14. The microfluidic platform of claim 13, wherein the gel channel comprises cells selected from the group consisting of fibroblasts, stromal cells, smooth muscle cells, astrocytes, pericytes, organ cells, pluripotent or multipotent cells and tumor cells.

15. The microfluidic platform of claim 13, wherein the chamber openings provide interstitial flow to the cells therein.

16. The microfluidic platform of claim 1 comprising one or more vascularized tissues and/or vascularized tissue masses in the gel chamber.

17. The microfluidic platform of claim 1 in combination with an MMP-2 inhibitor.

18. A method of forming vascularized tissue and/or vascularized tissue masses, the method comprising
seeding a gel channel of the microfluidic platforms of claim 1 containing one or more extracellular matrix components with endothelials cells, and
flowing medium through the gel channel to simulate interstitial flow.

19. The method of claim 18, wherein endothelial cells and stromal or fibroblast cells are seeded in one or both of the at least two tissue zones of the gel channel containing extracellular matrix.

20. The method of claim 18 comprising controlling the flow rate of the medium to increase perfusability of the tissue formed by the endothelial cells.

21. The method of claim 18, wherein different cells are seeded into each of the at least two tissue zones.

22. The method of claim 18, wherein the gel channel is seeded with cells selected from the group consisting of endothelial cells, stromal cells, smooth muscle cells, pericytes, fibroblasts, progenitor cells, and combinations thereof.

23. The method of claim 18, wherein the one or more extracellular matrix components are selected from the group consisting of fibrous proteins, hyaluronic acid, and proteoglycans, preferably collagen, fibrin, fibronectin, elastins, and laminin

24. The method of claim 18, wherein the mix of cells and one or more extracellular matrix components comprises tissue masses selected from the group consisting of tumors, organoids, spheroids, patient biopsy, and combinations thereof and cells forming vasculature.

25. The method of claim 20, wherein flowing medium through the gel channel comprises flow controlled by hydraulic pressure applied to a culture medium.

26. The method of claim 18 comprising administering an MMP-2 inhibitor.

27. The method of claim 18 comprising forming a blood brain barrier equivalent.

28. The method of claim 20, further comprising culturing the cells and one or more extracellular matrix components in the hydrogel chamber for a period between about 2 and 10 days.

29. A perfusable hydrogel vascularized tissue formed by the method of claim 18.

30. A method of enhancing growth and perfusability of vascularized tissue in culture comprising applying interstitial flow, preferably in combination with gel-liquid interface.

31. The method of claim 30 comprising administering an MMP2-inhibitor with the interstitial flow.
